(19) United States
(12) Patent Application Publication
(10) Pub. No.: US 2017/0367841 A1
(43) Pub. Date: Dec. 28, 2017

(54) Spinal Cages and Instruments for Inserting Same

(71) Applicant: CeramTec GmbH, Plochingen (DE)

(72) Inventors: Heinrich WECKER, Eckental (DE); Alfons KELNBERGER, Röthenbach (DE); Mateusz JUSZCZYK, Plochingen (DE); Rebecca MURRAY, Lauf (DE); Nikolas WILLMANN, Ulm (DE)

(21) Appl. No.: 15/536,589

(22) PCT Filed: Dec. 16, 2015
(86) PCT No.: PCT/EP2015/079918

§ 371 Date: Jun. 15, 2017

(30) Foreign Application Priority Data
Dec. 16, 2014 (DE) 10 2014 226 036.1

(51) Int. Cl. A61F 2/44; A61F 2/30; A61B 17/02; A61L 27/10

(52) U.S. Cl. CPC A61F 2/447; A61F 2/30767; A61B 2017/0256; A61L 27/10; A61F 2002/30003

(57) ABSTRACT

A spinal implant comprising an upper and a lower end plate connected by inner and outer lateral walls, said outer lateral walls forming an anterior wall, a posterior wall and two longitudinal walls, said inner lateral walls encompassing a receiving space, and engagement elements being arranged on and projecting from said upper and lower end plates. According to the invention, in order to avoid wear and abrasion when implanted and to improve ingrowth of the spinal implant into the tissue, said spinal implant consists of a ceramic material and carries a porous ceramic foam in at least some sub-sections.

The invention relates to different embodiments of spinal cages having associated insertion instruments. A spinal cage shall be construed to mean a spinal implant that may be used as a spinal disc replacement for fusing vertebrae.

Spinal cages for fusing vertebrae are known. Their geometry is adapted to the anatomy of the human vertebra, they are disposed between two vertebrae, and they replace all or part of the disc.

Typically, due solely to their mechanical properties, in a first phase of their disposition in the human body they hold the vertebrae at a distance and thus in an anatomically correct position. In a second phase they promote fusing of, and thus the growth of, the two vertebrae surrounding them.

Known components for fusing vertebrae are based on metal materials such as e.g. tantalum or titanium, plastics such as e.g. highly crosslinked PE materials (polyethylene) or PEEK (polyether ether ketone), or silicon nitride.

The underlying object of the invention is to improve a spinal implant according to the preamble to claim 1 such that while implanted no wear and no abrasion occur and said implant in-grows into the tissue better.

According to the invention, this object is attained using the features of claim 1.

Since the spinal implant comprises a ceramic material and carries a porous ceramic foam, at least in sub-sections, when implanted there is no wear and no abrasion and said implant in-grows into the tissue better.

The engaging elements are preferably teeth, grooves, or edges so that the implant anchors better.

Arranged on the anterior wall is at least one longitudinal hole that connects the inner lateral wall to the outer lateral wall. This longitudinal hole is the insertion opening for an insertion instrument. It is also possible for two longitudinal holes, spaced apart from one another, to be arranged on the anterior wall.

The two longitudinal holes are preferably arranged the same distance from the depth axis and are arranged on either side of said depth axis. This facilitates insertion with an insertion instrument, i.e., there is no jamming.

The two longitudinal holes preferably converge, i.e. they are arranged at an acute angle to the depth axis. The insertion instrument can grip the implant better because of this.

In one preferred embodiment, the upper end plate and the lower end plate run parallel to one another or the upper end plate and the lower end plate are at an angle to the center plane and the end plates drop continuously from the anterior wall to the posterior wall with respect to the center plane. Because of this the implant is better adapted to the natural shape of the vertebrae.

In one embodiment, the upper end plate and the lower end plate are arranged at an angle between 0° and 10°, preferably 2° to 5°, with respect to the center plane.

In one embodiment, the upper end plate runs at an angle to the center plane that is half as big as the angle between the lower end plate and the center plane. This facilitates better anchoring between the vertebrae.

The upper end plate preferably runs at an angle of 4° to the center plane, and the lower end plate at an angle of 2° to the center plane. Because of this the implant is better adapted to the natural shape of the vertebrae.

In a spinal implant having a longitudinal axis, a depth axis, and a width axis, wherein the depth axis and the width axis intersect the longitudinal axis, the spinal implant is preferably embodied essentially in a rectangle from the anterior wall to the width axis and from the width axis to the posterior wall the two lateral walls turn from the width axis at an angle between 60° and 70°.

Preferably present on the spinal implant are indentations that are filled with the ceramic foam and/or the ceramic foam is applied directly to the surfaces of the spinal implant. The indentations form a receiving space for the ceramic foam.

Preferably an L-shaped indentation that reaches to the anterior wall is disposed on each of the two longitudinal walls. These indentations are also contact points for the insertion instrument.

In one embodiment, the indentations are arranged on the upper end plate and on the lower end plate, and engaging elements, preferably edges, are disposed between the indentations. Due to this, both indentations and edges are arranged. The edges facilitate anchoring immediately following implantation until the implant is in-grown due to the ceramic foam.

In one embodiment, the upper end plate is arched.

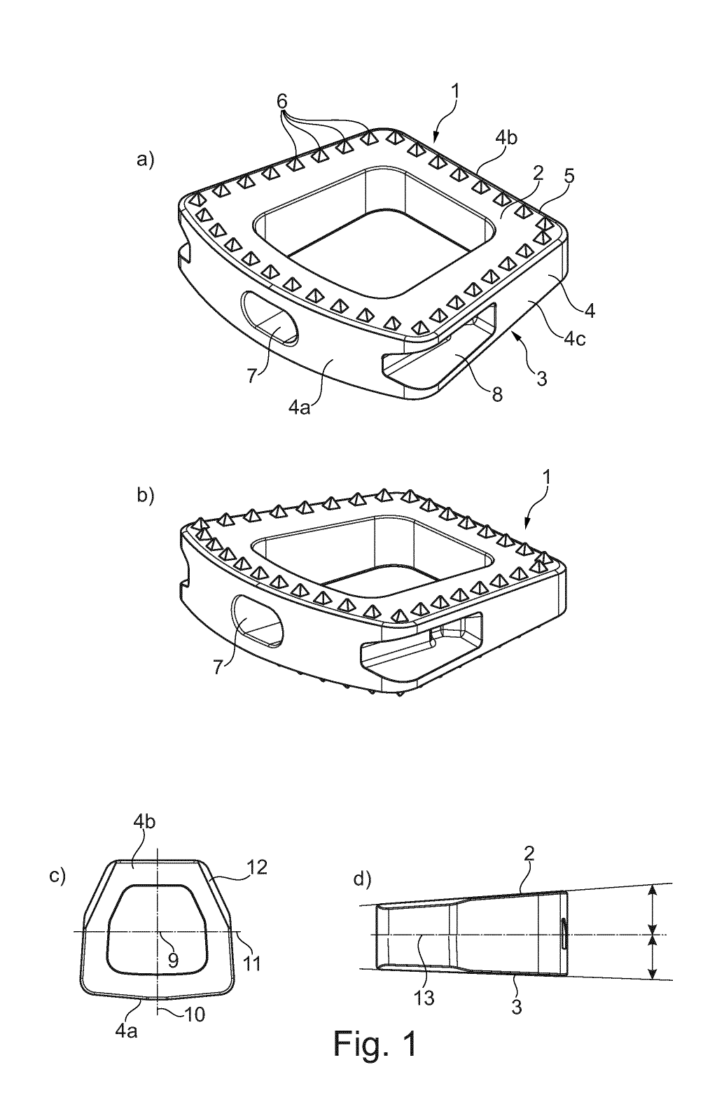

FIGS. 1a and 1b depict a first embodiment of an inventive spinal cage 1. This spinal cage 1 is embodied in a trapezoidal shape with a planar upper end plate 2 and a planar lower end plate 3 that are connected by lateral walls 4. Disposed adjacent to the outer edge 5 of the two end plates 2, 3 are teeth 6 that project from the end plates 2, 3 and facilitate better anchoring with the human vertebrae. These teeth 6 may also be arranged at different sites on the end plate 2, 3 and with a different quantity of teeth. Other structures, such as e.g. grooves or edges, may also be used instead of the teeth 6.

The lateral walls 4 are divided into an anterior wall 4a, posterior wall 4b, and two longitudinal walls 4c. A longitudinal hole 7 is disposed in the center of the anterior wall 4a. This longitudinal hole 7 is provided for the insertion of an explantation instrument, which shall be described later.

An L-shaped indentation 8 for engaging with an insertion instrument, which shall be described later, is disposed on each of the two longitudinal walls 4c. The indentation 8 leads to the anterior wall 4a, so that the insertion instrument does not project out of the indentation 8 when inserted.

FIG. 1c depicts the described spinal cage 1 from above. The depth axis 10 and the width axis 11 lead through the longitudinal axis 9. The spinal cage 1 is essentially embodied in a rectangular shape from the anterior wall 4a to the width axis 11. From the width axis 11 to the posterior wall 4b, the two lateral walls 4c turn from the width axis 11 at an angle between 60° and 70°. Optional chamfers 12 are disposed on the edge of the end plates 2, 3 from the width axis 11 to the posterior wall 4b. The teeth are not shown in this figure or in the following FIG. 1d.

FIG. 4d is a schematic depiction of a longitudinal side 4c, with no indentation. The upper end plate 2 runs at an angle of 4° to the center plane 13 and the lower end plate 3 runs at an angle of 2° to the center plane 13. Angles between 0° and 10°, preferably between 2° to 5°, are possible. The end plates 2, 3 thus drop continuously from the anterior wall 4a to the posterior wall 4b with respect to the center plane 13.

The trapezoidal shape is consistent with anatomy (uncinate processes). The total angle is divided asymmetrically (4° and 2°, in this case), but may also be divided symmetrically (e.g., 3° and 3°). This preferably applies for planar end plates 2, 3.

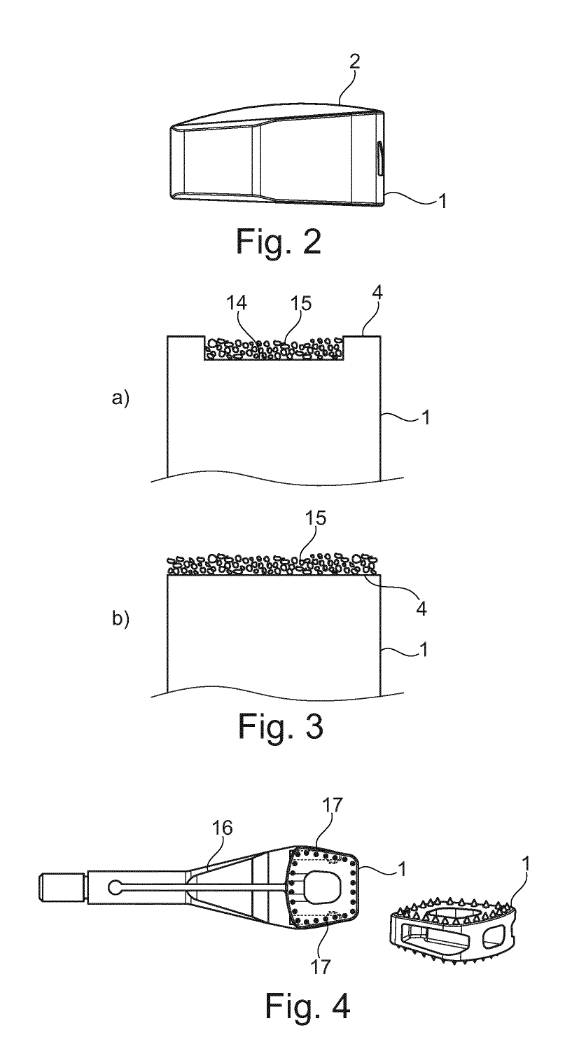

FIG. 2 depicts a variant of an inventive spinal cage 1 having a curved upper end plate 2, depicted schematically. This is an adaptation to the natural curvature of the lower vertebral end plate.

FIG. 3a depicts a segment from a lateral wall 4. Present on it is an indentation 14 that is filled with a ceramic foam 15. In this way, a porous surface that effects better in-growth of the bone is obtained. The indentation 14 and thus the ceramic foam 15 are preferably arranged circumferentially on the lateral walls 4.

FIG. 3b depicts a segment from a lateral wall 4. No indentation is present thereon, but instead the ceramic foam 15 is applied directly to the planar surface. In this way, as well, a porous surface is obtained that effects better in-growth of the bone. The ceramic foam 15 is preferably arranged circumferentially on the lateral walls 4.

Additional spinal cages and insertion instruments shall be described in the following.

FIG. 4 depicts the spinal cage 1 according to FIG. 1 and an associated insertion instrument 16. The insertion instrument 16 has fork tines 17 that engage in the indentations 8 (see FIG. 1) and in the lateral channels. The fork tines are stressed, so that a non-positive connection is created using the channels or V-channels (V-belt principle). Because of this the insertion instrument 16 does not catch when it is retracted. High leveraging forces may be transmitted using the interface, which is disposed far to the outside. The limited tensile forces are a drawback. An adapted insertion instrument 16 is required for each cage width. The view of the spinal cage 1 is also limited. The insertion instrument 16 covers the anterior wall 4a of the spinal cage 1.

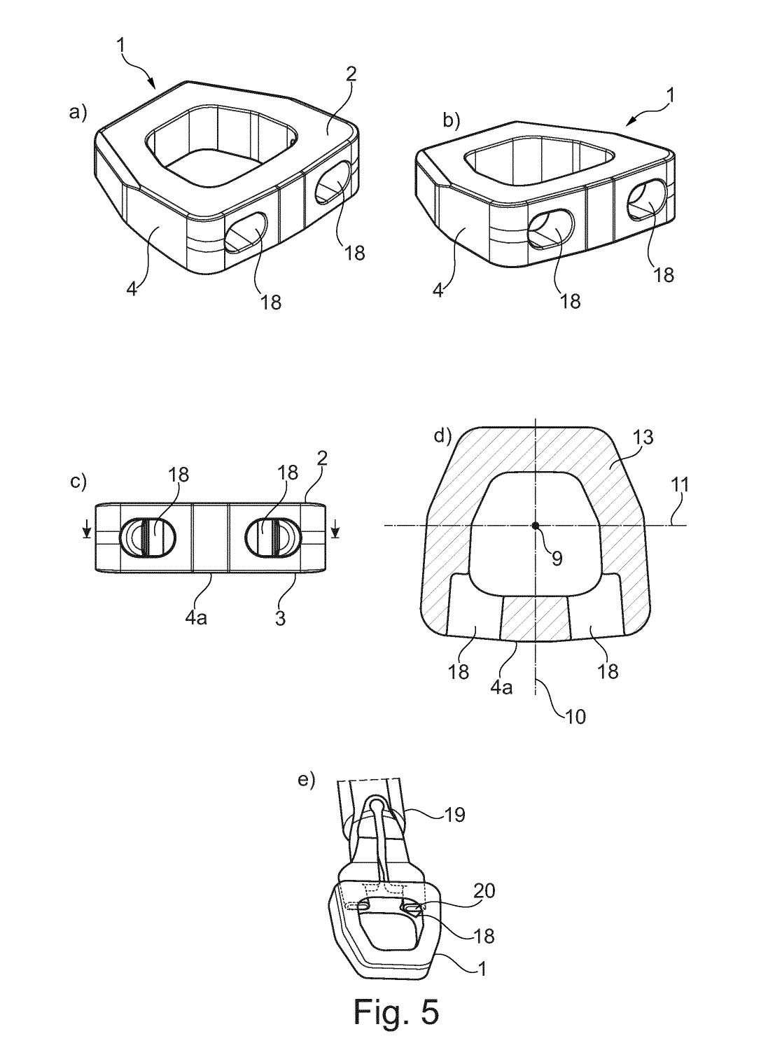

FIGS. 5a, 5b, 5c, 5d, 5d depict another inventive spinal cage 1. FIGS. 5a, 5b depict the spinal cage 1 in two perspective elevations. FIG. 5c is a view from the front, i.e., onto the anterior wall 4a, and FIG. 5d is a section along the center plane 13. FIG. 5e depicts this spinal cage 1 together with the appropriate insertion instrument 19.

In contrast to the spinal cage 1 from FIG. 1, the spinal cage 1 does not have any lateral indentations 8, nor does it have a center longitudinal hole 7. Otherwise, except from two oval longitudinal holes 18, it is identical to the spinal cage 1 from FIG. 1.

These longitudinal holes 18 are disposed, spaced apart from one another, in the anterior wall 4a. An important feature is that these two longitudinal holes 18 converge, i.e., they are arranged at an acute angle to the depth axis 10. The depth axis 10 and the width axis 11 intersect the longitudinal axis 9.

FIG. 5e depicts this spinal cage 1 together with the gripper or the tong-like insertion instrument 19. The insertion instrument 19 has two spreading arms 20 that engage in the longitudinal holes 18 and are then spread. The two spreading arms 20 are preferably adapted to the longitudinal holes 18 and also converge like them. This creates a positive fit. The longitudinal hole 18 and spreading arm 20 interface is independent of the size of the spinal cage 1. Only the limited contact surface between the spreading arms 20 and the longitudinal hole 18 is disadvantageous. The spinal cage 1 from FIG. 5e is only shown schematically.

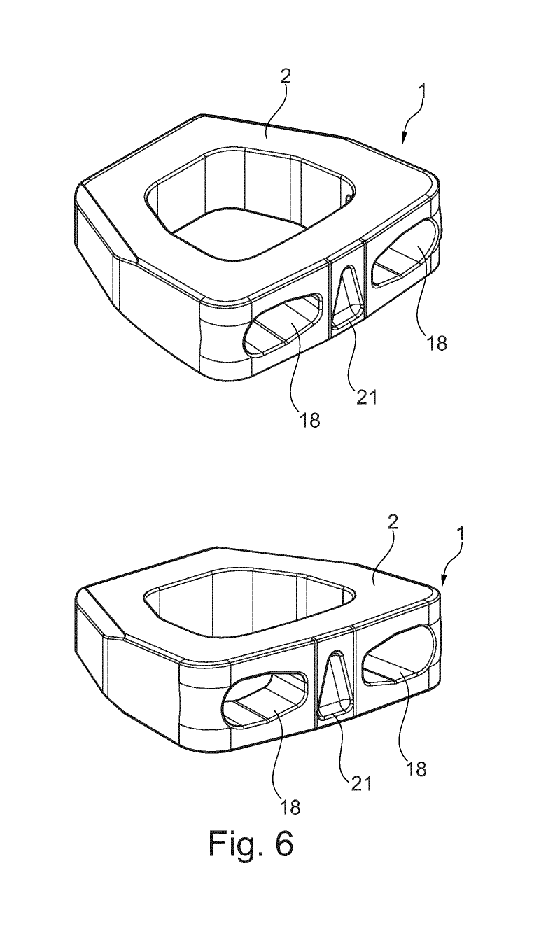

FIG. 6 depicts another embodiment of the inventive spinal cage 1. This spinal cage 1 is distinguished from the spinal cages 1 described in the foregoing by the shape of the longitudinal holes 18 and a recess arranged between the longitudinal holes.

In contrast to the spinal cage 1 in FIG. 1, the spinal cage 1 does not have any lateral indentations 8, nor does it have any center longitudinal hole 7. In this embodiment, it is important that the longitudinal holes are embodied in a V shape. This has the advantage that there is a larger support surface for the insertion instrument and thus better force transmission is possible. A recess 21 like an arrow may optionally be provided between the longitudinal holes 18. The arrow or the recess 21 indicates to the surgeon the direction of insertion and also acts as an engaging element for an insertion instrument.

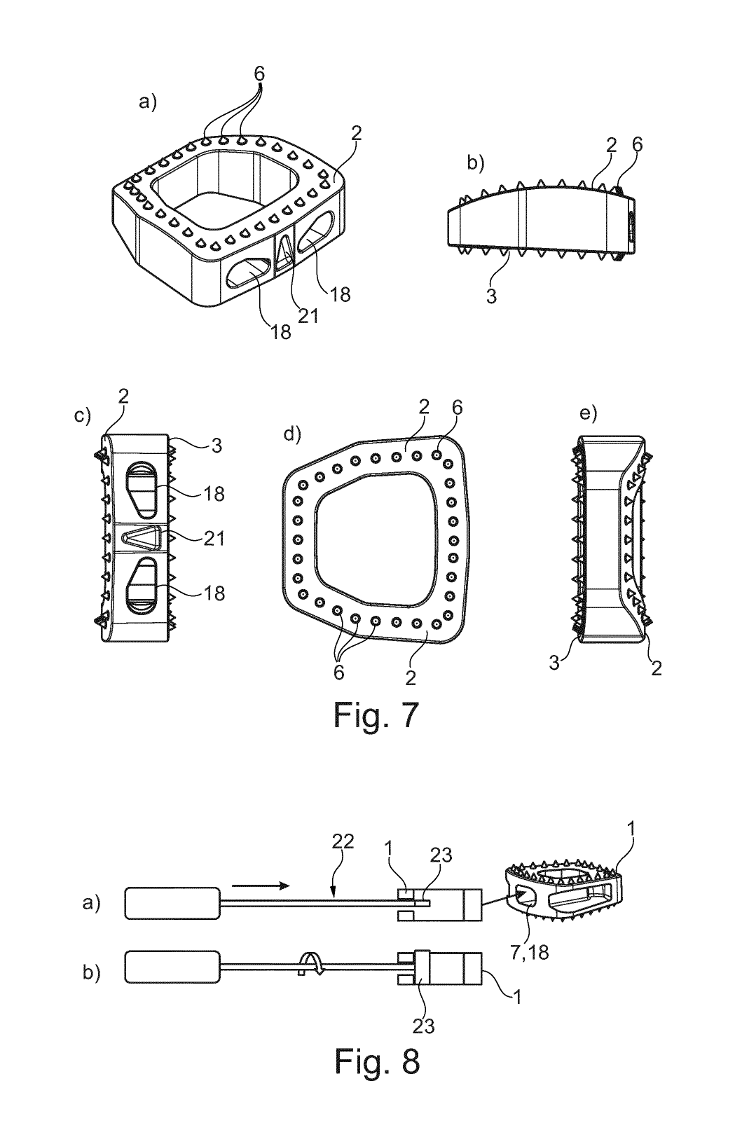

FIG. 7 depicts another embodiment of an inventive spinal cage 1. FIG. 7a is a perspective elevation of the spinal cage 1. It is easy to see that this spinal cage 1 does not have any lateral indentations 8, nor does it have a center longitudinal hole 7. What is important in this embodiment is that the longitudinal holes 18 are embodied in a trapezoidal shape (asymmetrically). Because of this, the wall of the longitudinal hole 18 that faces the lower end plate 3 is longer, i.e., has an even larger support surface for the tool. A clear orientation of the spinal cage 1 on the instrument is attained due to the recess 21. In addition, due to the embodiment of the longitudinal hole 18 as a trapezoid, it is not possible to insert the insertion instrument 18 incorrectly.

FIG. 7b depicts the spinal cage 1 from the side. It may be seen that the upper end plate 2 is arched. On the other hand, the lower end plate 3 is embodied flat. As FIG. 7b depicts, teeth 6 are arranged on the end plates 2 here, as well.

FIG. 7c depicts the spinal cage 1 from the anterior side with the trapezoidal longitudinal holes 18 and the recess 21. FIG. 7d depicts the upper end plate 2 with the teeth 6. FIG. 7e depicts the spinal cage 1 from the posterior side.

FIG. 8 depicts the spinal cage 1 according to FIG. 1 together with an explantation instrument 22. This explantation instrument 22 is provided with a hammer head 23 and is inserted into the longitudinal hole 7 or the longitudinal holes 18 and locked by rotating 90°. Then it is simple to explant the spinal cage 1. FIG. 8a depicts the explantation instrument 22 inserted in the spinal cage 1 and not yet locked. FIG. 8b depicts the explantation instrument 22 inserted into the spinal cage 1 and locked.

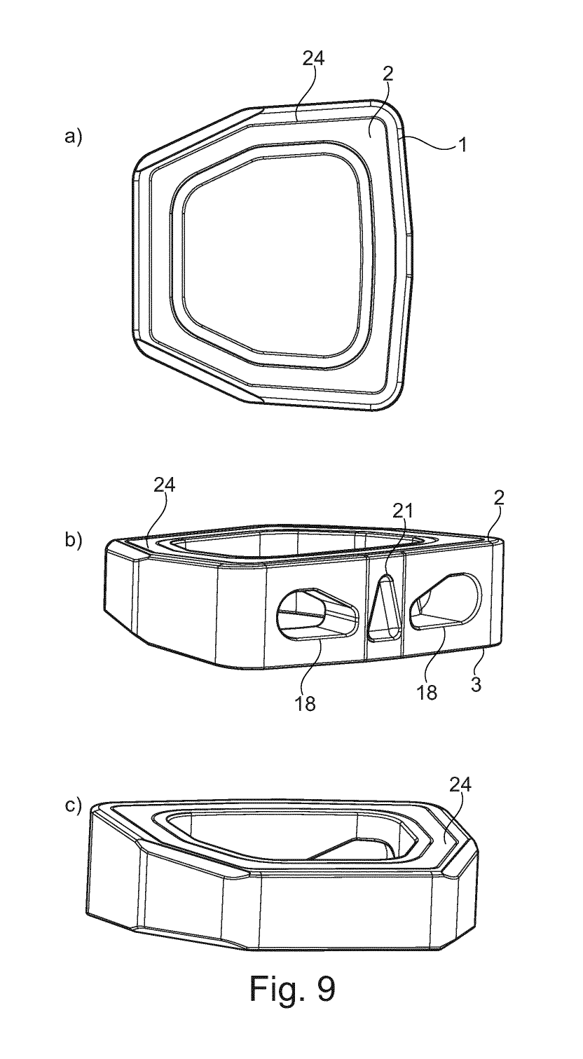

FIG. 9a depicts a spinal cage 1 having a circumferential groove 24 added to the upper end plate 2. As also described for FIG. 3, this groove 24 may be filled with a ceramic foam (not depicted in FIG. 9). This filling would look like that in FIG. 3. FIG. 9b depicts the spinal cage 1 from the anterior side with the longitudinal holes 18 and the recess 21 and FIG. 9c depicts the spinal cage 1 from the posterior side.

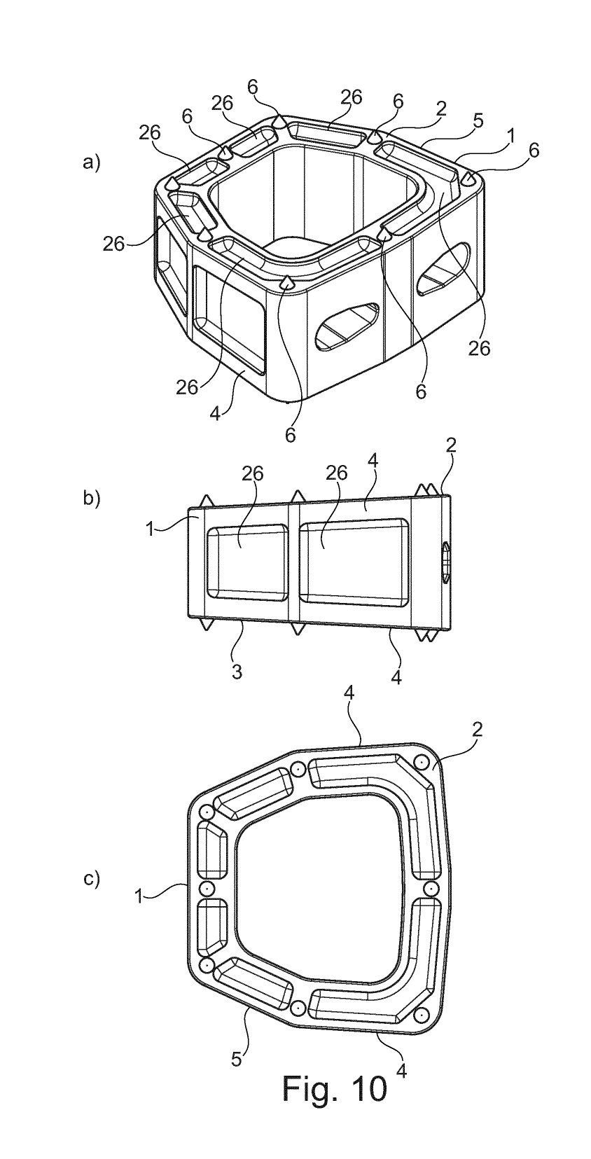

FIGS. 10a, 10b, and 10c depict an inventive spinal cage 1. This spinal cage 1 is embodied in a trapezoidal shape having an upper end plate 2 and a lower end plate 3 that are connected by lateral walls 4. Disposed adjacent to the outer edge 5 of both end plates 2, 3 are teeth 6 that project from the end plates 2, 3 and facilitate better anchoring with the human vertebrae. These teeth 6 may also be arranged at different sites on the end plates 2, 3 and with a different quantity of teeth. Other structures, such as e.g. grooves or edges, may also be used instead of the teeth 6.

FIG. 10a is a perspective elevation of the spinal cage 1, FIG. 10b depicts a view onto one side 4, and FIG. 10c depicts a view from above onto the upper end plate 2. The surfaces between the teeth 6 are provided with grooves 26 that may be filled with a ceramic foam (not shown in FIG. 10).

The lateral walls 4 are also provided with grooves 26 that may be filled with a ceramic foam (not shown in FIG. 10).

1. Spinal implant having an upper end plate and a lower end plate that are connected by inner lateral walls and outer lateral walls, wherein the outer lateral walls form an anterior wall, a posterior wall, and two longitudinal walls, the inner lateral walls surround a receiving space, and arranged on the upper end plate and lower end plate are engaging elements projecting therefrom, wherein the spinal implant comprises a ceramic material and carries a porous ceramic foam, at least in sub-sections.

2. Spinal implant according to claim 1, wherein the engaging elements are teeth, grooves, or edges.

3. Spinal implant according to claim 1, wherein at least one longitudinal hole that connects the inner lateral wall to the outer lateral wall is arranged on the anterior wall.

4. Spinal implant according to claim 3, wherein two longitudinal holes spaced apart from one another are arranged on the anterior wall.

5. Spinal implant according to claim 3, wherein the two longitudinal holes are arranged the same distance from the depth axis and are arranged on either side thereof.

6. Spinal implant according to claim 3, wherein the two longitudinal holes converge, i.e. they are arranged at an acute angle to the depth axis.

7. Spinal implant according to claim 1 having a center plane, wherein the upper end plate and the lower end plate run parallel to one another or the upper end plate and the lower end plate run at an angle to the center plane and the end plates drop continuously from the anterior wall to the posterior wall with respect to the center plane.

8. Spinal implant according to claim 7, wherein the upper end plate and the lower end plate are arranged at an angle between 0° and 10°, preferably between 2° and 5°, with respect to the center plane.

9. Spinal implant according to claim 8, wherein the upper end plate runs at an angle to the center plane that is half as big as the angle between the lower end plate and the center plane.

10. Spinal implant according to claim 8, wherein the upper end plate runs at an angle of 4° to the center plane and the lower end plate runs at an angle of 2° to the center plane.

11. Spinal implant according to claim 1 having a longitudinal axis, a depth axis, and a width axis, wherein the depth axis and the width axis intersect the longitudinal axis, wherein the spinal implant is embodied essentially in a rectangle from the anterior wall to the width axis and from the width axis to the posterior wall the two lateral walls turn from the width axis at an angle between 60° and 70°.

12. Spinal implant according to claim 1, wherein present on the spinal implant are indentations that are filled with the ceramic foam and/or the ceramic foam is applied directly to the surfaces of the spinal implants.

13. Spinal implant according to claim 1, wherein an L-shaped indentation that reaches to the anterior wall is disposed on each of the two longitudinal walls.

14. Spinal implant according to claim 12, wherein indentations are arranged on the upper end plate and on the lower end plate, and engaging elements that are preferably edges are disposed between the indentations.

15. Spinal implant according to claim 1, wherein the upper end plate is arched.
